(19) United States
(12) Patent Application Publication
(10) Pub. No.: US 2025/0000331 A1
(43) Pub. Date: Jan. 2, 2025

(54) ENDOSCOPIC EXAMINATION SUPPORT SYSTEM, ENDOSCOPIC EXAMINATION SUPPORT METHOD, AND STORAGE MEDIUM

(71) Applicant: OLYMPUS MEDICAL SYSTEMS CORP., Tokyo (JP)

(72) Inventors: Jun HANE, Tokyo (JP); Kensuke MIYAKE, Tokyo (JP)

(73) Assignee: OLYMPUS MEDICAL SYSTEMS CORP., Tokyo (JP)

(21) Appl. No.: 18/884,386

(22) Filed: Sep. 13, 2024

Related U.S. Application Data
(63) Continuation of application No. PCT/JP2022/011682, filed on Mar. 15, 2022.

(51) Int. Cl. A61B 1/00; A61B 5/00

(52) U.S. Cl. CPC A61B 1/000096; A61B 1/00045; A61B 5/4255

(57) ABSTRACT

An examination time period evaluator acquires a time evaluation index related to a predetermined examination step (e.g. a net observation step) in an endoscopic examination. An image classifier acquires an implementation record of the endoscopic examination. An examination time period classifier calculates an actual time, i.e. a time required for the predetermined examination step, based on the implementation record. A display controller may display the evaluation index and the actual time on a display apparatus. The display controller may further display a comparison result between the evaluation index and the actual time on the display apparatus.

### CROSS-REFERENCE TO RELATED APPLICATIONS

This application is based upon and claims the benefit of priority from the International Application No. PCT/JP2022/011682, filed on Mar. 15, 2022, the entire contents of which are incorporated.

### BACKGROUND

### 1. Technical Field

The present disclosure relates to an endoscopic examination support system, an endoscopic examination support method, and a storage medium.

### 2. Description of the Related Art

In major endoscopic societies, a removal time period of 6 minutes or longer is recommended as an index of a sufficient observation time period in a case without treatment. In relation to this, there has been proposed a method of classifying an endoscopic examination time period into a non-observation time period, a normal observation time period, a detailed observation time period, and a treatment time period at the time of endoscopic examination and recording the classified result.

### SUMMARY

An endoscopic examination support system according to one aspect of the present disclosure includes one or more processors having hardware. The processor acquires a time evaluation index related to a predetermined examination step in an endoscopic examination, acquires an implementation record of the endoscopic examination, and calculates an actual time, i.e. a time required for the predetermined examination step, based on the implementation record.

Another aspect of the present disclosure is an endoscopic examination support method. The method acquires a time evaluation index related to a predetermined examination step in an endoscopic examination, acquires an implementation record of the endoscopic examination, and calculates an actual time, i.e. a time required for the predetermined examination step, based on the implementation record.

Note that arbitrary combinations of the above components and modifications of the expressions of the present disclosure among methods, apparatuses, systems, recording media, computer programs, and the like are also effective as aspects of the present disclosure.

### BRIEF DESCRIPTION OF THE DRAWINGS

FIG. 1 is a diagram illustrating an overall system configuration related to large intestine endoscopy according to an embodiment;

FIG. 2 is a diagram illustrating an example of an endoscope used in the embodiment;

FIG. 3 is a diagram illustrating a configuration example of an endoscopic examination support system according to the embodiment;

FIG. 4 is a table summarizing removal time period classification results according to Specific Example 1;

FIG. 5 is a diagram illustrating a graph in which the removal time period classification results according to Specific Example 1 are classified based on the insertion length;

FIG. 6 is a table summarizing removal time period classification results according to Specific Example 2;

FIG. 7 is a diagram illustrating a graph in which the removal time period classification results according to Specific Example 2 are classified based on the elapsed time at the time of removal;

FIG. 8 is a table summarizing classification results obtained by classifying net observation time periods according to Specific Example 1 based on the intestinal tract state;

FIGS. 9A and 9B are views illustrating specific examples of endoscopic images of the intestinal tract with almost no folds;

FIGS. 10A to 10C are views illustrating specific examples of endoscopic images of the intestinal tract with many deep folds;

FIGS. 11A to 11D are views illustrating specific examples of endoscopic images of the intestinal tract with diverticula;

FIG. 12 is a table in which evaluation results are added to the classification results of the net observation time periods shown in FIG. 8;

FIG. 13 is a table in which the removal time period classification results according to Specific Example 1 are classified for each site;

FIG. 14 is a diagram illustrating a graph in which the removal time period classification results shown in FIG. 13 are classified for each site; and

FIG. 15 is a flowchart illustrating an example of a movement of the endoscopic examination support system according to the embodiment.

### DETAILED DESCRIPTION

The invention will now be described by reference to the preferred embodiments. This does not intend to limit the scope of the present invention, but to exemplify the invention.

The present embodiment relates to large intestine endoscopy. Generally, in large intestine endoscopy, observation and treatment are performed while an endoscope is inserted into the cecum and then removed toward the anus. One of quality indicators (QIs) in large intestine endoscopy is a removal time period. To ensure the quality of lesion screening in large intestine endoscopy, it is desirable to present a direct index (e.g. an observation rate of the intestinal tract surface) that the large intestinal mucosa has been observed fully and completely. However, from the viewpoint of a technical difficulty level, major endoscopic societies recommend, as an indirect index, a removal time period of 6 minutes or longer in a case without treatment.

However, when the index is limited to the case without treatment, many cases are excluded from the target. Particularly, in a case where, after a simple examination proves positive, large intestine endoscopy is performed to conduct a detailed examination at a hospital, the proportion of cases excluded from the target is considerably increased. Further, the proportion of the target cases decreases as the number of reexaminations increases and the number of treatments increases in a large hospital. Consequently, it is difficult to ensure the quality of lesion screening for all cases.

Meanwhile, the removal time period includes, in addition to the observation time period for lesion screening, a detailed examination time period for examining a site suspected of being a lesion, a treatment time period for removing the lesion, and a washing/suction time period for washing and suctioning residues and moisture. It is not necessarily determined that the observation time period for lesion screening is sufficiently secured only when the removal time period is 6 minutes or longer. Thus, there is a need for a mechanism for objectively and accurately determining that the observation time period is sufficiently secured.

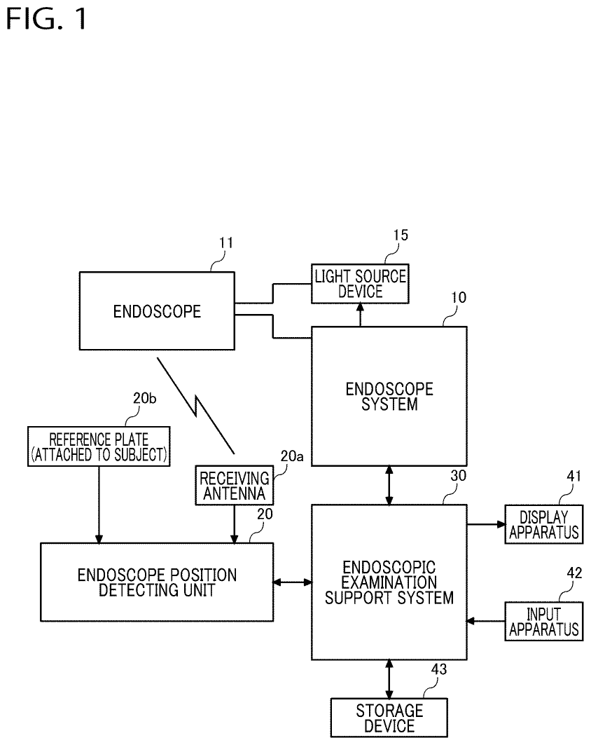

FIG. 1 is a diagram illustrating an overall system configuration related to large intestine endoscopy according to an embodiment. In the present embodiment, an endoscope system 10, an endoscope 11, a light source device 15, an endoscope position detecting unit (UPD) 20, an endoscopic examination support system 30, a display apparatus 41, an input apparatus 42, and a storage device 43 are used. The endoscope 11 according to the present embodiment is a large intestine endoscope that is inserted into the large intestine of a subject (patient).

The endoscope 11 includes a lens and a solid-state imaging device (e.g. a CMOS image sensor, a CCD image sensor, or a CMD image sensor). The solid-state imaging device converts light condensed by the lens into an electrical signal and outputs an endoscopic image (the electrical signal) to the endoscope system 10. The endoscope 11 includes a forceps channel. A practitioner (physician) can perform various treatments during an endoscopic examination by passing a treatment tool through the forceps channel.

The light source device 15 includes a light source such as a xenon lamp, and supplies observation light (such as white light, narrow band light, fluorescence, or near infrared light) to the distal end of the endoscope 11. The light source device 15 also includes a pump that feeds water or air to the endoscope 11.

The endoscope system 10 controls the light source device 15 and processes an endoscopic image input from the endoscope 11. The endoscope system 10 has, for example, functions such as narrow band imaging (NBI), red dichromatic imaging (RDI), texture and color enhancement imaging (TXI), and extended depth of field (EDOF).

In the narrow band imaging, irradiation with light of specific wavelengths of purple (415 nm) and green (540 nm) strongly absorbed by hemoglobin in blood makes it possible to acquire an endoscopic image in which capillaries and microstructures in the superficial portion of the mucous membrane are emphasized. In the red dichromatic imaging, irradiation with light of specific wavelengths of three colors (green, amber, and red) makes it possible to acquire an endoscopic image in which the contrast of the deep tissue is emphasized. In the texture and color enhancement imaging, an endoscopic image is generated in which three elements of “structure”, “color tone”, and “brightness” of the mucosal surface under normal light observation are optimized. In the extended depth of field, two images at near and far distances come into focus, these images are combined, and thus it possible to acquire an endoscopic image with a wide focal range.

The endoscope system 10 outputs an endoscopic image obtained by processing an endoscopic image input from the endoscope 11 or an endoscopic image input from the endoscope 11 as it is to the endoscopic examination support system 30.

The endoscope position detecting unit 20 is a device for observing a three-dimensional shape of the endoscope 11 inserted into the lumen of the subject. A receiving antenna 20a is connected to the endoscope position detecting unit 20. The receiving antenna 20a is an antenna for detecting a magnetic field generated by a plurality of magnetic coils built in the endoscope 11.

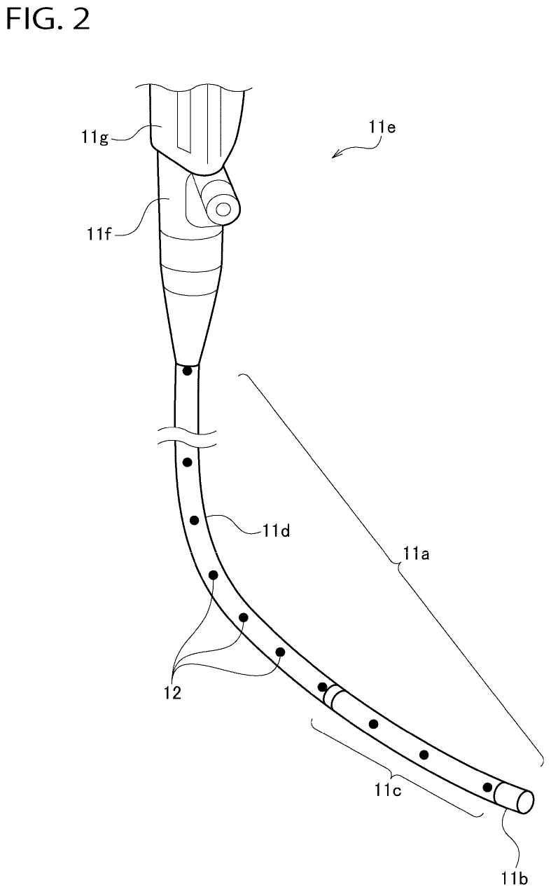

FIG. 2 is a diagram illustrating an example of the endoscope 11 used in the embodiment. The endoscope 11 includes an elongated tubular insertion portion 11a formed of a flexible member and an operation unit 11e connected to a base end of the insertion portion 11a. The insertion portion 11a has a distal end rigid portion 11b, a bending portion 11c, and a flexible tube 11d, in order from the distal end side to the base end side. A base end of the distal end rigid portion 11b is connected to a distal end of the bending portion 11c, and a base end of the bending portion 11c is connected to a base end of the flexible tube 11d.

The operation unit 11e includes a main body 11f from which the flexible tube 11d extends and a grip 11g connected to a base end of the main body 11f. The grip 11g is gripped by the practitioner. A universal cord extends from the operation unit 11e. The universal cord includes an imaging electric cable, a light guide, and the like, which extend from the inside of the insertion portion 11a, and is connected to the endoscope system 10 and the light source device 15.

The distal end rigid portion 11b is a distal end of the insertion portion 11a and is also a distal end of the endoscope 11. The distal end rigid portion 11b incorporates a solid-state imaging device, an illumination optical system, an observation optical system, and the like. The illumination light emitted from the light source device 15 is propagated to the distal end surface of the distal end rigid portion 11b along the light guide, and is emitted from the distal end surface of the distal end rigid portion 11b toward the observation target in the lumen.

The bending portion 11c is formed by connecting joint rings along the longitudinal axis direction of the insertion portion 11a. The bending portion 11c is bent in a desired direction depending on the practitioner's operation input to the operation unit 11e, and the position and direction of the distal end rigid portion 11b change depending on the bent state of the bending portion.

The flexible tube 11d is a tubular member extending from the main body 11f of the operation unit 11e, has desired flexibility, and is bent by an external force. The practitioner inserts the insertion portion 11a into the large intestine of the subject while bending the bending portion 11c or twisting the flexible tube 11d.

A plurality of magnetic coils 12 is arranged inside the insertion portion 11a at predetermined intervals (e.g. an interval of 10 cm) along the longitudinal direction. When a current is supplied, each of the magnetic coils 12 generates a magnetic field. The plurality of magnetic coils 12 each functions as a position sensor for detecting respective positions of the insertion portion 11a.

Now description will be returned to FIG. 1. The receiving antenna 20a receives magnetic fields transmitted from the plurality of magnetic coils 12 built in the insertion portion 11a of the endoscope 11, and outputs the magnetic fields to the endoscope position detecting unit 20. The endoscope position detecting unit 20 applies the intensity of the magnetic field of each of the plurality of magnetic coils 12 received by the receiving antenna 20a to a predetermined position detection algorithm and estimates the three-dimensional position of each of the plurality of magnetic coils 12. The endoscope position detecting unit 20 generates a three-dimensional endoscope shape of the insertion portion 11a of the endoscope 11 by performing curve interpolation on the estimated three-dimensional positions of the plurality of magnetic coils 12.

A reference plate 20b is attached to the subject (e.g. the abdomen of the subject). A posture sensor for detecting the posture of the subject is arranged on the reference plate 20b. As the posture sensor, for example, a three-axis acceleration sensor or a gyro sensor can be used. In FIG. 1, the reference plate 20b is connected to the endoscope position detecting unit 20 by a cable, and the reference plate 20b outputs three-dimensional posture information indicating the posture (i.e. the posture of the subject) of the reference plate 20b to the endoscope position detecting unit 20.

Note that a plurality of magnetic coils similar to the plurality of magnetic coils 12 built in the insertion portion 11a of the endoscope 11 may be used as the posture sensor arranged on the reference plate 20b. In this case, the receiving antenna 20a receives magnetic fields transmitted from the plurality of magnetic coils arranged on the reference plate 20b, and outputs the magnetic fields to the endoscope position detecting unit 20. The endoscope position detecting unit 20 applies the intensity of the magnetic fields of each of the plurality of magnetic coils received by the receiving antenna 20a to a predetermined posture detection algorithm and generates three-dimensional posture information indicating the posture of the reference plate 20b (i.e. the posture of the subject).

The endoscope position detecting unit 20 changes the generated three-dimensional endoscope shape following the change in the three-dimensional posture information. Specifically, the endoscope position detecting unit 20 changes the three-dimensional endoscope shape so as to cancel the change in the three-dimensional posture information. As a result, even when the posture of the subject is changed during the endoscopic examination, it is possible to always recognize the endoscope shape from a specific viewpoint (e.g. a viewpoint of viewing the abdomen of the subject vertically from the front side of the abdomen).

The endoscope position detecting unit 20 can acquire an insertion length indicating a length of a portion of the endoscope 11 inserted into the large intestine and an elapsed time after insertion of the endoscope 11 into the large intestine (hereinafter, referred to as “insertion time period”). For example, the endoscope position detecting unit measures the insertion length using the position at the timing when the practitioner inputs the examination start operation to the input apparatus 42 as a base point, and measures the insertion time period using the timing as a starting point. Note that the endoscope position detecting unit 20 may estimate the position of the anus from the generated three-dimensional endoscope shape and the difference in magnetic field intensity between the magnetic coil inside the body and the magnetic coil outside the body, and may use the estimated position of the anus as the base point of the insertion length.

To measure the insertion length with high accuracy, an encoder may be placed near the anus of the subject. The endoscope position detecting unit 20 detects the insertion length using the position of the anus as a base point, based on a signal from the encoder.

The endoscope position detecting unit 20 adds the insertion length and the insertion time period to the three-dimensional endoscope shape after posture correction based on the three-dimensional posture information, and outputs the three-dimensional endoscope shape to the endoscopic examination support system 30.

The endoscopic examination support system 30 generates support information for endoscopic examination based on an endoscopic image input from the endoscope system and an endoscope shape input from the endoscope position detecting unit 20, and presents the support information to the practitioner. Further, the endoscopic examination support system 30 generates endoscopic examination history information based on the endoscopic image input from the endoscope system 10 and the endoscope shape input from the endoscope position detecting unit 20, and records the endoscopic examination history information in the storage device 43.

The display apparatus 41 includes a liquid crystal monitor and an organic EL monitor, and displays an image input from the endoscopic examination support system 30. The input apparatus 42 includes a mouse, a keyboard, a touch panel, and the like, and outputs operation information input by the practitioner or the like to the endoscopic examination support system 30. The storage device 43 includes a storage medium such as an HDD or an SSD, and stores the endoscopic examination history information generated by the endoscopic examination support system 30. The storage device 43 may be a dedicated storage device attached to the endoscope system 10, a database on an in-hospital server connected via an in-hospital network, or a database in a cloud server.

In the system configuration illustrated in FIG. 1, the reference plate 20b can be omitted. Further, the endoscope position detecting unit 20 and the receiving antenna 20a can also be omitted. In a case where the insertion length or the insertion time period is measured by a device other than the endoscope position detecting unit 20 and the endoscope shape is not used in the process of identifying the site to be described later, the endoscope position detecting unit 20, the receiving antenna 20a, and the reference plate 20b can be omitted.

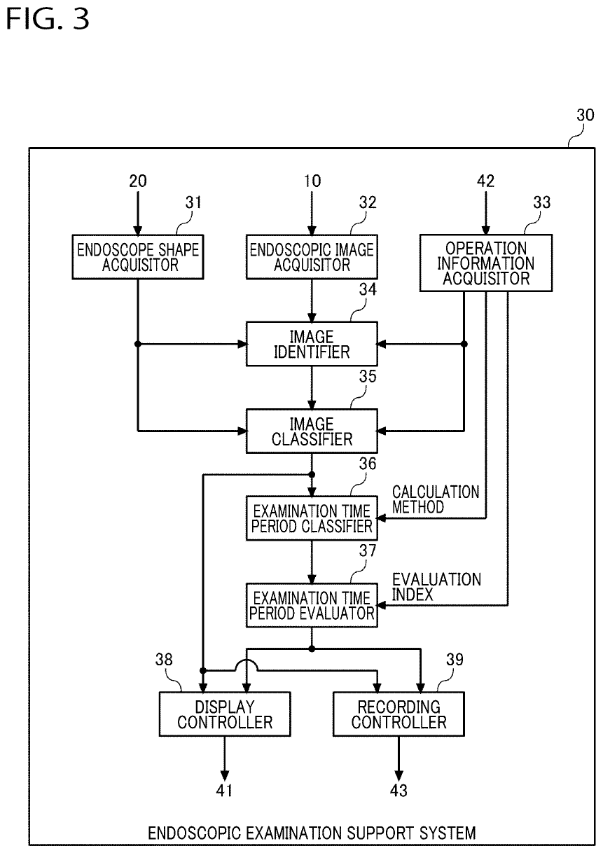

FIG. 3 illustrates a configuration example of the endoscopic examination support system 30 according to the embodiment. The endoscopic examination support system 30 may be constructed with a processing apparatus dedicated to endoscopic examination support, or may be constructed with a general-purpose server (which may be a cloud server). Alternatively, the endoscopic examination support system 30 may be constructed by an arbitrary combination of a processing apparatus dedicated to endoscopic examination support, a general-purpose server (which may be a cloud server), and an apparatus dedicated to image diagnosis. Alternatively, the endoscopic examination support system 30 may be integrally constructed with the endoscope system 10.

The endoscopic examination support system 30 includes an endoscope shape acquisitor 31, an endoscopic image acquisitor 32, an operation information acquisitor 33, an image identifier 34, an image classifier 35, an examination time period classifier 36, an examination time period evaluator 37, a display controller 38, and a recording controller 39. These components can be realized by at least one arbitrary processor (e.g. CPU or GPU), memory (e.g. DRAM), or other large-scale integrations (LSIs) (e.g. FPGA and ASIC) in terms of hardware, and are realized by a program or the like loaded in a memory in terms of software. Here, functional blocks realized by cooperation of hardware and software are illustrated. Therefore, it is understood by those skilled in the art that these functional blocks can be implemented in various forms by only hardware, only software, or a combination of hardware and software.

The endoscope shape acquisitor 31 acquires an endoscope shape from the endoscope position detecting unit 20. The endoscope shape also includes information on an insertion length and an insertion time period. The endoscopic image acquisitor 32 acquires an endoscopic image from the endoscope system 10.

The image identifier 34 includes a plurality of machine learning models for detecting a site/state of an examination target organ (large intestine in the present embodiment), a treatment tool, an observation condition, a lesion, and the like from the endoscopic image. The plurality of machine learning models is generated by machine learning in which a large number of endoscopic images including annotations such as various sites/states, various treatment tools, various observation conditions, and various lesions are used as a supervised data set. The annotations are added by an annotator having specialized knowledge of a physician or the like. For example, CNN, RNN, or LSTM, which is a type of deep learning, can be used for machine learning.

The site of the large intestine is roughly classified into rectum, sigmoid colon, descending colon, transverse colon, ascending colon, and cecum in order from the anus side. The image identifier 34 can input the endoscopic images to a site learning model and detect the site of the large intestine from the endoscopic images. At that time, the image identifier 34 may identify the site based on detection results of a plurality of endoscopic images continuous in time series. For example, when an identical site is detected in frames with a set number or more in endoscopic images of 30 or 60 consecutive frames, the image identifier 34 identifies the site as a formal detection site.

In addition, the image identifier 34 may identify the site in consideration of the anteroposterior relationship of the detection site or the endoscope shape acquired from the endoscope position detecting unit 20. For example, the image identifier 34 identifies whether the moving direction of the endoscope 11 is an insertion direction (from the anus to the cecum) or a removal direction (from the cecum to the anus). In the case of the insertion direction, the image identifier 34 switches the detection site from the descending colon to the transverse colon when the left colic flexure is detected, and switches the detection site from the transverse colon to the ascending colon when the right colic flexure is detected. In the case of the insertion direction, the image identifier 34 switches the detection site from the ascending colon to the transverse colon when the right colic flexure is detected, and switches the detection site from the transverse colon to the descending colon when the left colic flexure is detected.

In addition, the image identifier 34 may improve the accuracy of the detection of the site in consideration of the three-dimensional position of the distal end rigid portion 11b (hereinafter, referred to as “endoscope distal end”) based on the endoscope shape acquired from the endoscope position detecting unit 20. For example, in a case where the position of the endoscope distal end estimated from the endoscope shape and the position of the detection site based on the image identification are inconsistent, the image identifier 34 discards the detection result based on the image identification.

In addition, the image identifier 34 can input an endoscopic image to a learning model for a treatment tool/suction tube and detect the treatment tool (e.g. biopsy forceps or snare) or the suction tube from the endoscopic image. Further, the image identifier 34 can input an endoscopic image to a learning model for residue, cleaning foam, bleeding, and moisture, and detect the residue, cleaning foam, bleeding, or moisture from the endoscopic image. The image identifier 34 may identify each of the treatment tool, the suction tube, the residue, the cleaning foam, the bleeding, and the moisture in consideration of the practitioner's operation information input to the operation unit 11e of the endoscope 11 or the input apparatus 42.

In addition, the image identifier 34 can input an endoscopic image to an observation condition learning model and identify the observation condition from the endoscopic image. For example, the image identifier 34 can identify whether normal light or special light (e.g. narrow band light or red light) is used. The image identifier 34 can identify, for example, the presence or absence of a staining solution and the type of the staining solution when the staining solution is used. The image identifier 34 can identify, for example, the presence or absence of image enhancement, and the type and intensity of image enhancement when image enhancement is used. The image identifier 34 can identify, for example, the presence or absence of zooming and the zooming magnification when zooming is used. Note that the image identifier 34 may identify various observation conditions using the practitioner's operation information input to the operation unit 11e of the endoscope 11 or the input apparatus 42 or in consideration of the operation information.

In addition, the image identifier 34 can input an endoscopic image to a learning model for an intraluminal state and determine the state in the lumen from the endoscopic image. The image identifier 34 can detect, for example, the presence or absence of folds of a predetermined height or more and the presence or absence of diverticula. Further, the image identifier 34 can input an endoscopic image to a lesion learning model and detect a lesion candidate from the endoscopic image.

Note that the image identifier 34 may check the quality of the endoscopic image prior to the identification of the image of the detection target. The image identifier 34 excludes the endoscopic image determined to have poor image quality (e.g. blurring, out of focus, and luminance abnormality (e.g. halation)) from the image identification target as the detection target.

The image classifier 35 identifies endoscopic images at the time of reaching the deepest portion and at the time of completion of removal. The deepest portion in large intestine endoscopy is normally the cecum. For example, the image classifier 35 determines the endoscopic image when the cecum is detected by the image identifier 34 as the endoscopic image at the time of reaching the deepest portion. Alternatively, the image classifier 35 may determine the endoscopic image when the insertion length acquired from the endoscope position detecting unit 20 becomes the longest as the endoscopic image at the time of reaching the deepest portion. Alternatively, the image classifier 35 may determine the endoscopic image at the timing of inputting of the insertion completion operation to the input apparatus 42 by the practitioner as the endoscopic image at the time of reaching the deepest portion. Depending on the practitioner, the endoscope 11 may be inserted into the ileum. Further, depending on the subject, the endoscope 11 is not able to be inserted into the cecum, and the ascending colon may be the deepest portion in large intestine endoscopy.

The image classifier 35 determines the endoscopic image at the timing of switching from the image inside the body to the image outside the body as the endoscopic image at the time of completion of removal. Here, the image classifier 35 may determine the endoscopic image at the timing of inputting of the removal completion operation to the input apparatus 42 by the practitioner as the endoscopic image at the time of completion of removal.

The image classifier 35 classifies a plurality of endoscopic images imaged between the time of reaching the deepest portion and the time of completion of removal under various conditions. The image classifier 35 executes lesion screening classification, detailed examination classification, treatment classification, washing/suction classification, observation condition classification, intraluminal state classification, image quality classification, and the like on the plurality of endoscopic images.

In the lesion screening classification, the image classifier 35 classifies an endoscopic image in which the practitioner is estimated to be performing an operation (procedure) other than the detailed examination and treatment as an endoscopic image at the time of lesion screening. The operation other than the treatment includes washing and suction. The endoscopic image related to the operation other than the detailed examination, treatment, and washing and suction, which is estimated not to have been subjected to lesion screening, is also excluded from the endoscopic image at the time of lesion screening. Note that the image classifier 35 may consider the use of observation with special light when identifying the endoscopic image at the time of lesion screening.

In the detailed examination classification, the image classifier 35 classifies an endoscopic image remaining at a site including a lesion candidate for a predetermined period of time or longer as an endoscopic image at the time of detailed examination. When the endoscopic image at the time of detailed examination is identified, the image classifier 35 may consider the improvement in zooming magnification, the use of observation with special light, or the use of observation by staining.

In the treatment classification, the image classifier 35 classifies an endoscopic image in which the treatment tool is detected as an endoscopic image at the time of treatment. In the washing/suction classification, the image classifier 35 classifies an endoscopic image in which the suction tube, the residue, the cleaning foam, the bleeding, or the moisture is detected as an endoscopic image at the time of washing/suction. Washing and suction during observation are performed to wash and suck the residue and the moisture to make the surface of the lumen easy to observe. Also, washing and suction are performed to wash away the staining solution with water. Washing and suction after the treatment are performed to wash away bleeding and dirt.

In the observation condition classification, the image classifier 35 identifies a light source setting condition, an image enhancement setting condition, and the like for each endoscopic image. The observation condition classification is performed independently of other image classifications. In the intraluminal state classification, the image classifier 35 identifies the presence or absence of folds of a predetermined height or more, the presence or absence of diverticula, and the presence or absence of other states or diatheses related to the difficulty level of the observation for each endoscopic image. The intraluminal state classification is also performed independently of other image classifications.

In the image quality classification, the image classifier 35 identifies the presence or absence of blurring, out of focus, and luminance abnormality (e.g. halation) for each endoscopic image, and excludes the endoscopic image in which these poor image qualities are detected from targets to be classified into respective examination steps.

In addition, the image classifier 35 can also classify a plurality of endoscopic images for each site. For example, the image classifier 35 classifies the plurality of endoscopic images for each site (e.g. rectum, sigmoid colon, descending colon, transverse colon, ascending colon, and cecum) obtained by anatomically classifying the large intestine. The image classifier 35 identifies a site of each endoscopic image based on at least one of the sites detected by image identification or the information on the position of the endoscope distal end of the endoscope shape supplied from the endoscope position detecting unit 20. The classification for each site is performed independently of other image classifications.

Furthermore, the image classifier 35 can classify the insertion length by a predetermined length (e.g. 5 cm). The image classifier 35 can acquire the insertion length from the endoscope position detecting unit 20. The classification by the insertion length is performed independently of other image classifications. Consequently, it is possible to confirm the time taken for lesion screening or the like for each specific site or specific position. Alternatively, it is possible to confirm the time rate of items including the operation state, the observation condition, the intraluminal state, and the like in a specific site or a specific position unit.

A user such as a physician can use the input device 42 to set an image classification method for the image classifier 35. The user can change the setting of the image classification method to meet the needs of the practitioner or the facility, recommendations of the society or the like, or legal requirements.

In this manner, the image classifier 35 acquires an implementation record of the endoscopic examination. The implementation record of the endoscopic examination includes a plurality of endoscopic images continuous in time series in the endoscopic examination. The classification method of the endoscopic examination includes classification based on the examination steps. The image classifier 35 classifies each of the endoscopic images included in the plurality of time-series endoscopic images as any one of the examination steps, based on image identification results or the like.

The examination time period classifier 36 calculates the actual time, i.e. the time required for each of the examination steps based on the result of classifying the implementation record of the endoscopic examination by the image classifier 35. A plurality of examination steps of the endoscopic examination includes a predetermined examination step. The predetermined examination step may be an observation step obtained by removing at least the detailed examination step and the treatment step from all the steps of the endoscopic examination. The predetermined examination step may be a net observation step obtained by removing the washing step and the suction step from the observation step.

The image classifier 35 can classify the plurality of endoscopic images by various manners, in addition to classification based on the examination steps. The examination time period classifier 36 can aggregate the actual time for each of the items including the classifications. Further, the examination time period classifier 36 can also calculate the time ratio between the items including the classifications. The user can set a method of calculating the actual time of the evaluation target item to examination time period classifier 36 through the input apparatus 42. The calculation method for display may be different from the calculation method for recording. For example, the calculation method for display may be simpler than the calculation method for recording.

The examination time period evaluator 37 acquires the actual time related to each of the examination steps calculated by the examination time period classifier 36. A user such as a physician can set an evaluation index of an actual time related to a predetermined examination step (e.g. the appropriate observation time period) to the examination time period evaluator 37 through the input apparatus 42. The examination time period evaluator 37 acquires an evaluation index of an actual time related to a predetermined examination step.

The examination time period evaluator 37 compares the actual time related to the predetermined examination step with the evaluation index related to the predetermined examination step, and evaluates the actual time related to the predetermined examination step based on the comparison result. The examination time period evaluator 37 can also evaluate an actual time other than the predetermined examination step. An evaluation index for determining whether the actual time of the evaluation target is sufficient, short, or too long can be set for each actual time of the evaluation target. As the evaluation index, a constant numerical value or a numerical value calculated from a mathematical expression is assumed. The evaluation index is desirably an evaluation index whose effectiveness has been statistically confirmed or an evaluation index recommended by academic societies. Values other than these indices, for example, a value whose effectiveness is being verified or a value temporarily set may be used. In a case where it is not desirable to set a value, no setting is possible. Meanwhile, the value setting is possible when it is considered that setting is desired.

The display controller 38 displays the actual time related to the predetermined examination step and the evaluation index related to the predetermined examination step on the display apparatus 41. The display controller 38 can further display a comparison result between the actual time and the evaluation index as an evaluation result on the display apparatus 41. The display controller 38 can also display the actual time of each of the plurality of examination steps of the endoscopic examination on the display apparatus 41.

The display controller 38 can display, on the display apparatus 41, the actual time and evaluation result related to a predetermined examination item, during or after the examination. Displaying the actual time and evaluation result related to the predetermined examination item in real time or substantially real time (delay of about several seconds) during the examination enables the practitioner to determine whether the lesion screening is sufficient in the examination room. In a case where the lesion screening is insufficient, the practitioner can perform at least a partial lesion screening again.

The recording controller 39 records, in the storage device 43, examination time period recorded data in which the actual time of each of the examination steps and at least the evaluation result of a predetermined examination step are comprehensively recorded. The recording of the examination time period recorded data in the storage device 43 may be temporary or long-term. The examination time period recorded data recorded in the storage device 43 can be transferred to another database in the hospital after the data format is appropriately changed. Further, the examination time period recorded data recorded in the storage device 43 can be displayed on the display apparatus as a reference source in response to reference from the outside. The transfer of the data to the outside or the display of the data in response to reference from the outside causes the practitioner or other medical personnel to refer to the examination time period recorded data after the examination.

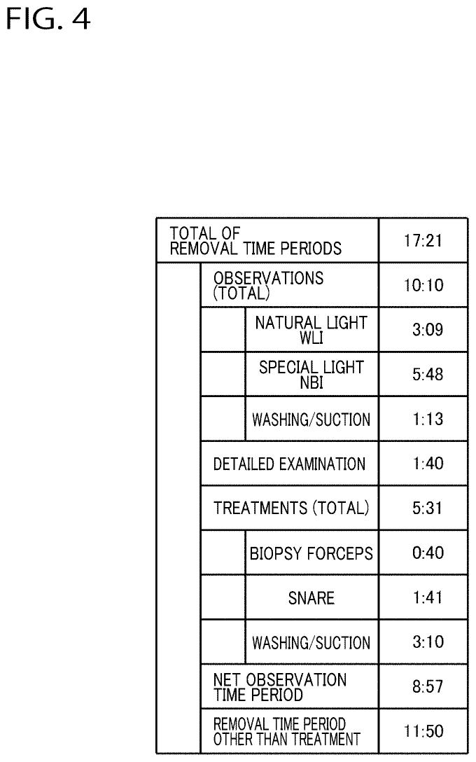

FIG. 4 is a table summarizing removal time period classification results according to Specific Example 1. The removal time period is defined by an elapsed time from the arrival at the deepest portion to the completion of removal. The examination time period classifier 36 roughly classifies the removal time period into three large groups: observation time period, detailed examination time period, and treatment time period. The examination time period classifier 36 classifies the observation time period into three small groups: natural light (WLI) observation time period, special light (NBI) observation time period, and washing/suction time period. The examination time period classifier 36 roughly classifies the treatment time period into three groups: biopsy forceps operating time period, snare operating time period, and washing/suction time period.

The examination time period classifier 36 calculates a net observation time period (8:57) by excluding, from the removal time period (17:21=17 min 21 s, the same applies hereinafter), the treatment time period (5:31), the detailed examination time period (1:40), and the washing/suction time period in the observation (1:13). The net observation time period (8:57) can also be obtained from the total of the natural light (WLI) observation time period (3:09) and the special light (NBI) observation time period (5:48). The examination time period classifier 36 can calculate the removal time period (11:50) other than the treatment by excluding the treatment time period (5:31) from the removal time period (17:21). Note that the calculation of the removal time period other than the treatment is optional.

In this manner, it is possible to measure the net observation time period used for lesion screening itself without the time period for ancillary operations such as washing and suction. The net observation time period can be measured in all cases. The removal time period other than the treatment can also be measured in all cases. Note that the actual time to be evaluated can be selected as appropriate, and a plurality of actual times may be selected as evaluation targets.

The display controller 38 can classify the actual time of each of the examination steps of the endoscopic examination for each segmentation obtained by dividing the insertion length of the endoscope 11 during the endoscopic examination at predetermined length intervals and display the classified actual time on the display apparatus 41. The insertion length is defined in a range of 0 [cm] to a length [cm] when the endoscope 11 reaches the deepest portion.

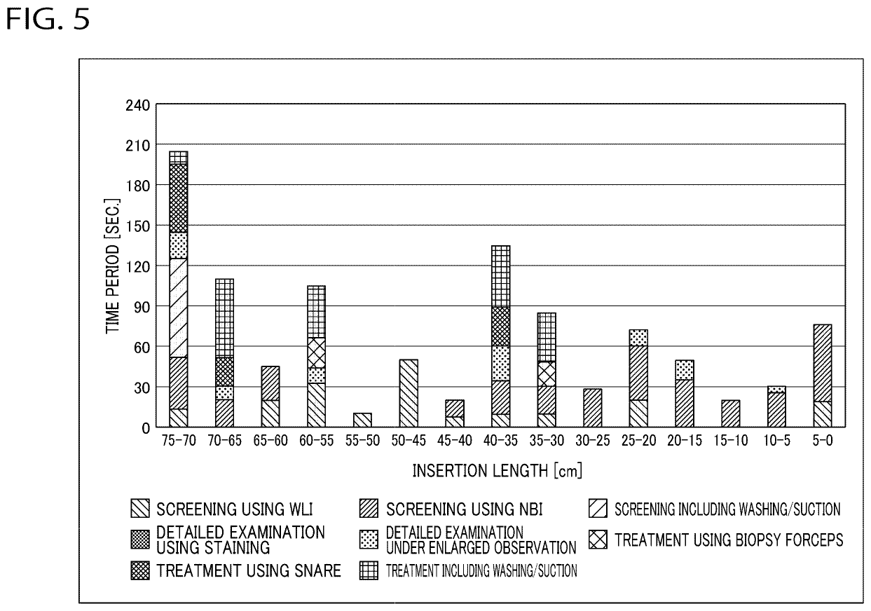

FIG. 5 is a diagram illustrating a graph in which the removal time period classification results according to Specific Example 1 are classified based on the insertion length. The horizontal axis represents the insertion length, and the breakdown of the removal time period is displayed as a bar graph in units of 5 cm. In the graph illustrated in FIG. 5, the detailed examination time period is further classified into a detailed examination time period using a staining solution and a detailed examination time period under enlarged observation. A user such as a physician can confirm the operation content and the observation time period for each position of the endoscope 11 with reference to the graph illustrated in FIG. 5.

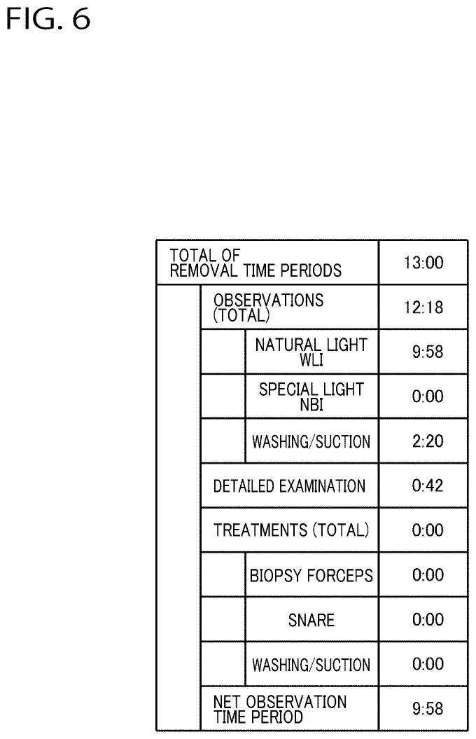

FIG. 6 is a table summarizing removal time period classification results according to Specific Example 2. The display controller 38 can classify the actual time of each of the examination steps of the endoscopic examination for each segmentation obtained by dividing the elapsed time at the time of removal during the endoscopic examination at predetermined time intervals, and display the classified time on the display apparatus 41.

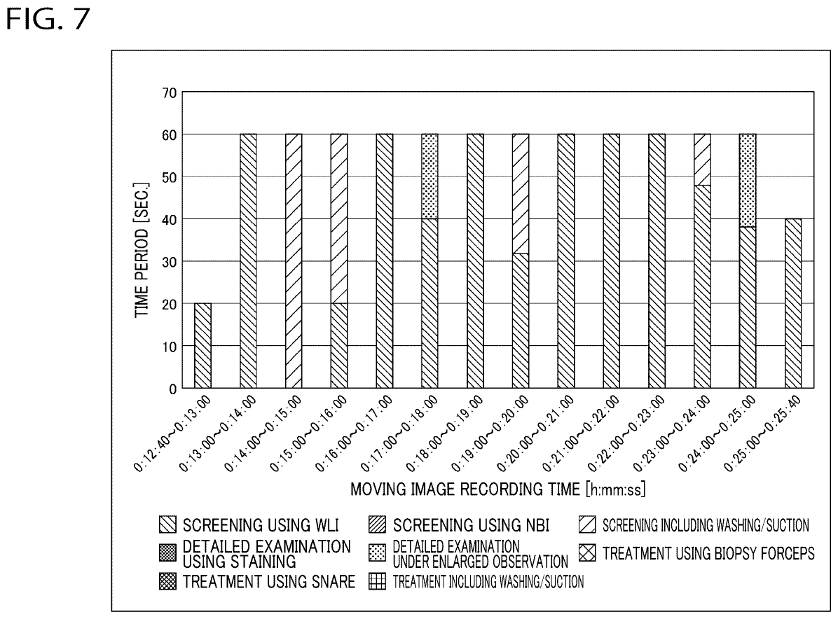

FIG. 7 is a diagram illustrating a graph in which the removal time period classification results according to Specific Example 2 are classified based on the elapsed time at the time of removal. The elapsed time at the time of removal corresponds to the moving image recording time by the endoscope 11, from the time of arrival at the deepest portion to the time of completion of removal. The horizontal axis represents the elapsed time, and the breakdown of the removal time period is displayed as a bar graph in units of 1 minute. In the example shown in FIG. 7, it can be seen that the detailed examination has been performed between 0:17:00 and 0:18:00 and between 0:24:00 and 0:25:00. In Specific Example 2, it takes 2:20 to perform washing and suction, and it takes 9:58 to perform observation with natural light (WLI).

Incidentally, the intestinal tract has a part in which lesion screening is easy and a part in which lesion screening is difficult. For example, a part with almost no folds can be easily observed, and the observation time period is relatively short. Meanwhile, in a part with many deep folds and an undulating part of the diverticulum, it takes a relatively long observation time to confirm whether there is a lesion in the shaded part. Accordingly, it is effective to confirm the observation time period of the part with almost no folds, the part with many deep folds, and the part with diverticula, in order to confirm whether or not the part in which lesion screening is difficult has been observed for a time commensurate with the difficulty.

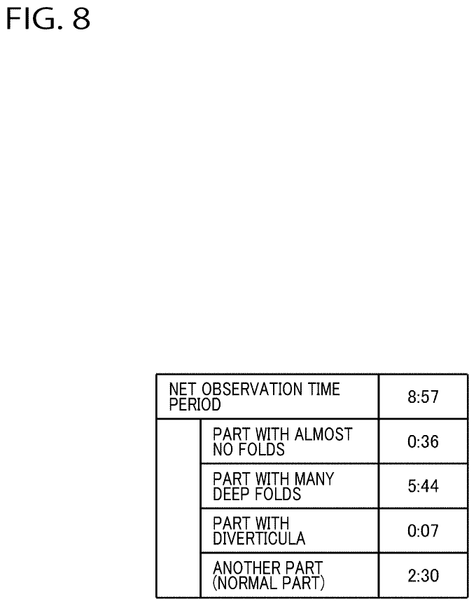

FIG. 8 is a table summarizing classification results obtained by classifying net observation time periods according to Specific Example 1 based on the intestinal tract state. The examples in FIG. 8 show examples in which the net observation time period (8:57) is classified into four groups: observation time period in the part with almost no folds (0:36), observation time period in the part with many deep folds (5:44), observation time period in the part with diverticula (0:07), and observation time period in another part (normal part) (2:30).

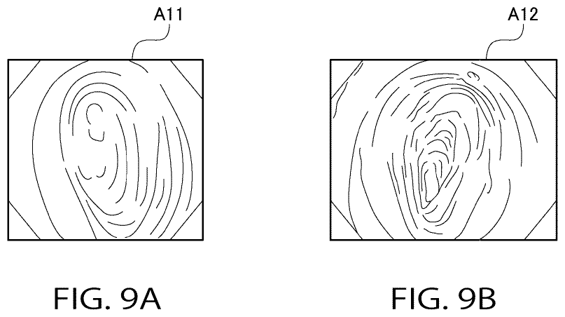

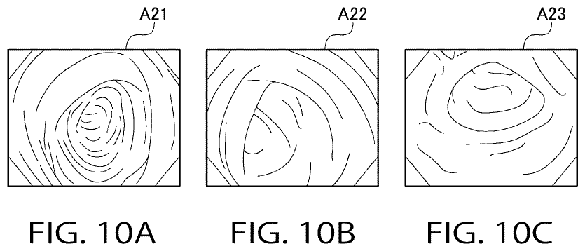

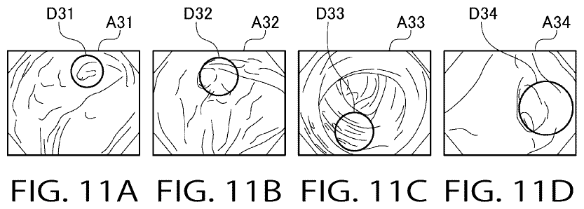

FIGS. 9A and 9B are views illustrating specific examples of endoscopic images A11 and A12 of the intestinal tract with almost no folds. FIGS. 10A to 10C are views illustrating specific examples of endoscopic images A21 to A23 of the intestinal tract with many deep folds. FIGS. 11A to 11D are views illustrating specific examples of endoscopic images A31 to A34 of the intestinal tract with diverticula D31 to D34.

The image classifier 35 classifies endoscopic images corresponding to the net observation step, which are included in the plurality of time-series endoscopic images, for each state of the examination target organ (the intestinal tract in the present embodiment) based on the image identification result by the image identifier 34. The examination time period classifier 36 calculates the actual time for each state in the net observation step based on the result classified for each state. The state of the intestinal tract is classified in accordance with the observation difficulty level. The state of the intestinal tract may include the state of folds of the intestinal tract. The state of the intestinal tract may include the presence or absence of diverticula.

The examination time period evaluator 37 acquires the actual time for each state in the net observation step calculated by the examination time period classifier 36. The examination time period evaluator 37 acquires an evaluation index for each state in the net observation step. The examination time period evaluator 37 compares the actual time for each state and the evaluation index for each state in the net observation step, and evaluates the actual time for each state in the net observation step based on the comparison results.

The display controller 38 displays, on the display apparatus 41, the actual time for each state in the net observation step and the evaluation index for each state in the net observation step. The display controller 38 can further display a comparison result between the actual time and the evaluation index as an evaluation result for each state on the display apparatus 41.

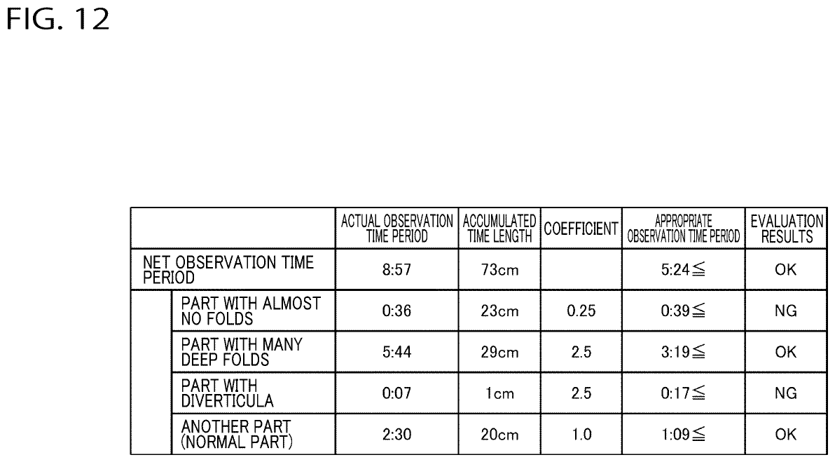

FIG. 12 is a table in which evaluation results are added to the classification results of the net observation time periods shown in FIG. 8. The accumulated time length corresponding to the net observation time period (8:57) is 73 cm, the accumulated time length of the part with almost no folds is 23 cm, the accumulated time length of the part with many deep folds is 29 cm, the accumulated time length of the part with diverticula is 1 cm, and the accumulated time length of another part (normal part) is 20 cm.

The examination time period classifier 36 calculates the accumulated time length for each state in the net observation step based on the result classified for each state of the endoscopic image corresponding to the net observation step. The examination time period evaluator 37 sets the evaluation index for each state in the net observation step in accordance with the accumulated time length for each state in the net observation step or in accordance with the accumulated time length for each state in the net observation step and the type of the state of the intestinal tract. For example, the examination time period evaluator 37 sets an appropriate observation time period for each state as the evaluation index for each state.

The examination time period evaluator 37 sets an appropriate observation time period for each state per unit length of the intestinal tract in a part with deep folds having a certain depth or more, in other words, a part with folds having a certain height or more, or a part in which the number of folds having a certain height or more is larger than the set value, to be longer than an appropriate observation time period for each state per unit length of the intestinal tract in the normal part. Further, the examination time period evaluator 37 sets an appropriate observation time period for each state per unit length of the intestinal tract in the part with diverticula to be longer than an appropriate observation time period for each state per unit length of the intestinal tract in the part with no diverticula.

In the example shown in FIG. 12, observing 70 cm of the normal part in 4 minutes (about 3.43 seconds per 1 cm) is set as the standard appropriate observation time period. The appropriate observation time period of each part classified for each state is obtained by multiplying the standard appropriate observation time period by a predetermined coefficient. The coefficient of the part where the observation difficulty level is higher than the normal part is set to a value exceeding 1.0, and the coefficient of the part where the observation difficulty level is lower than the normal part is set to a value less than 1.0. In the example shown in FIG. 12, a coefficient of the part with almost no folds is set to 0.25, and a coefficient of the undulating part (the part with many deep folds or the part with diverticula) is set to 2.5.

The final appropriate observation time period of each part can be obtained by multiplying the appropriate observation time period per unit length after correction based on the observation difficulty level by the accumulated time length of each part. In a case the correction based on the observation difficulty level is not performed, the final appropriate observation time period of each part can be obtained by multiplying the standard appropriate observation time period by the accumulated time length of each part.

In the case shown in FIG. 12, the appropriate observation time period of the entire net observation time period is 5:24 or longer. Further, the appropriate observation time period of the part with almost no folds is 0:39 or longer, whereas the actual observation time period of the part with almost no folds is 0:36. Accordingly, it can be seen that the actual observation time period of the part with almost no folds is slightly short. In the example shown in FIG. 12, OK is displayed in the item in which the actual observation time period is equal to or longer than the appropriate observation time period, and NG is displayed in the item in which the actual observation time period is shorter than the appropriate observation time period. Note that the examination time period evaluator 37 may also determine whether the actual observation time period of each part is excessively long.

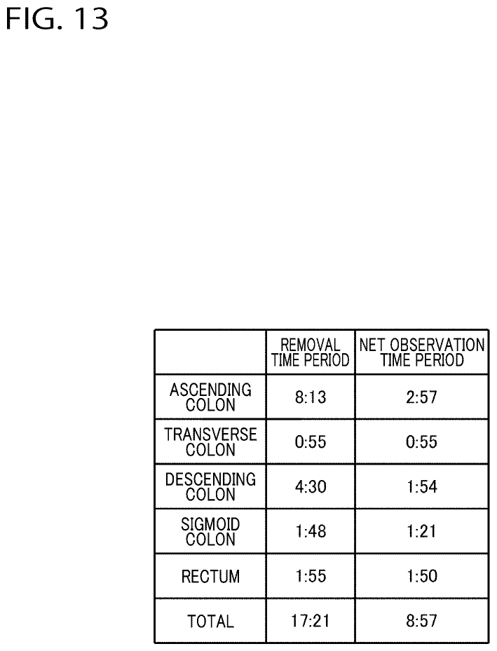

FIG. 13 is a table in which the removal time period classification results according to Specific Example 1 are classified for each site. As shown in FIG. 4, the removal time period according to Specific Example 1 is 17:21, and the net observation time period is 8:57. In the case of performing classification for each site, as shown in FIG. 13, the removal time period in the ascending colon is 8:13, the net observation time period is 2:57, the removal time period in the transverse colon is 0:55, the net observation time period is 0:55, the removal time period in the descending colon is 4:30, the net observation time period is 1:54, the removal time period in the sigmoid colon is 1:48, the net observation time period is 1:21, the removal time period in the rectum is 1:55, and the net observation time period is 1:50.

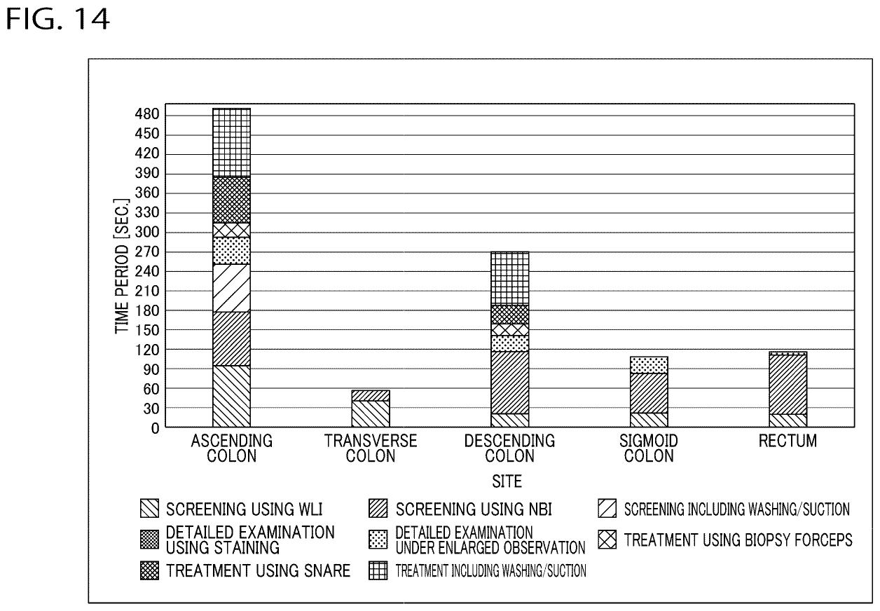

FIG. 14 is a diagram illustrating a graph in which the removal time period classification results shown in FIG. 13 are classified for each site. The horizontal axis represents the classification of the site, and the breakdown of the removal time period for each site is displayed as a bar graph. Further, as illustrated in FIG. 5, it is also possible to classify operation contents for each site.

The image classifier 35 classifies endoscopic images corresponding to the net observation step, which are included in the plurality of time-series endoscopic images, for each site of the examination target organ (the intestinal tract in the present embodiment) based on the image identification result by the image identifier 34, and the like. The examination time period classifier 36 calculates the actual time for each site in the net observation step based on the result classified for each site.

The examination time period evaluator 37 acquires the actual time for each site in the net observation step calculated by the examination time period classifier 36. The examination time period evaluator 37 acquires an evaluation index for each site in the net observation step. The examination time period evaluator 37 compares the actual time for each site and the evaluation index for each site in the net observation step, and evaluates the actual time for each site in the net observation step based on the comparison results.

The display controller 38 displays, on the display apparatus 41, the actual time for each site in the net observation step and the evaluation index for each site in the net observation step. The display controller 38 can further display a comparison result between the actual time and the evaluation index as an evaluation result for each site on the display apparatus 41.

A graph is generated in which the removal time period classification result is further classified for each site, and thus the removal time period and the net observation time period can be confirmed for each site. Consequently, more detailed analysis and determination, such as whether there is no uneven observation, can be performed. Hence, it is possible to analyze and determine where washing and suction were performed for a long time, where the treatment of the lesion was performed for a long time, and whether the observation time for each site was sufficient.

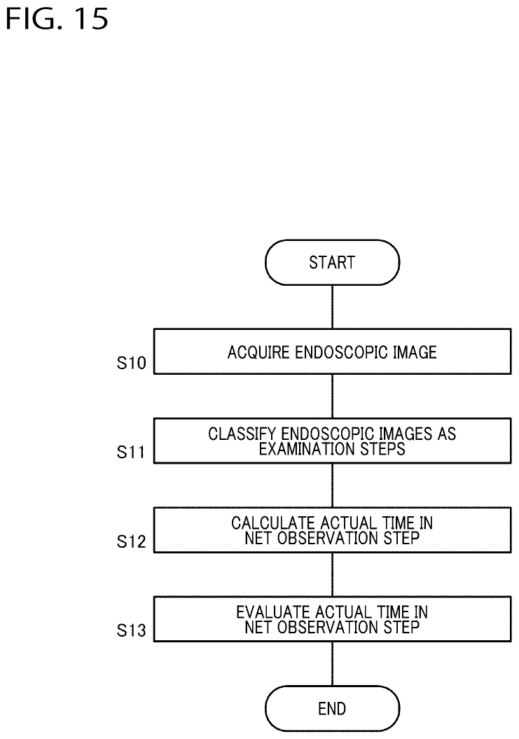

FIG. 15 is a flowchart illustrating an example of a movement of the endoscopic examination support system 30 according to the embodiment. The endoscopic image acquisitor 32 acquires a plurality of time-series endoscopic images from the endoscope system 10 (S10). The image classifier 35 classifies the acquired plurality of endoscopic images as the examination steps (S11). The examination time period classifier 36 calculates the actual time of the net observation step based on the endoscopic image classified as the net observation step (S12). The examination time period evaluator 37 compares the actual time of the net observation step with the appropriate observation time period, and evaluates the observation time period of the net observation step based on the comparison result (S13).

As described above, according to the present embodiment, it is possible to appropriately evaluate the quality of lesion screening by measuring the net observation time period in the endoscopic examination. In major endoscopic societies, a removal time period of 6 minutes or longer is recommended as an index of a sufficient observation time period in a case without treatment. However, the removal time period in the endoscopic examination also includes the treatment time period and the washing/suction time period. Thus, the index that the removal time period is 6 minutes or longer cannot guarantee that a sufficient observation time period is secured. Even in a case where the removal time period is 6 minutes or longer, taking a long time for the treatment or the washing/suction operation makes it impossible to secure a sufficient observation time period.

On the other hand, in the present embodiment, the net observation time period in which the treatment time period and the washing/suction time period are excluded from the removal time period is measured, and thus it is possible to appropriately evaluate whether or not a sufficient observation time period is secured. Further, the observation time period for each state or site of the intestinal tract is measured, and the measured observation time is compared with the appropriate observation time period for each state or site, and thus it is possible to more finely evaluate the quality of lesion screening.

The present disclosure has been described above based on the multiple embodiments. It is to be understood by those skilled in the art that these embodiments are merely examples, that various modifications can be made by combining the components and the processing processes, and that such modifications are also within the scope of the present disclosure.

In the above-described embodiment, an example has been described in which the endoscope position detecting unit is connected to the endoscopic examination support system 30, and the endoscopic examination support system 30 acquires an endoscope shape from the endoscope position detecting unit 20. In this respect, the present disclosure is also applicable to a system in which the endoscope position detecting unit 20 is omitted. In that case, the image classifier 35 cannot use the information related to the endoscope shape, and classifies the endoscopic image based on the image recognition result and the operation information. Note that in a case where an encoder is placed near the anus of the subject and the encoder is connected to the endoscopic examination support system 30, the endoscopic examination support system 30 can acquire an insertion length from the encoder.

What is claimed is:

1. An endoscopic examination support system comprising
one or more processors having hardware,
wherein the processors include:
an examination time period evaluation apparatus (37) that acquires an evaluation index for each state in which a time evaluation index related to a predetermined examination step in an endoscopic examination is defined for each state of an examination target organ in the endoscopic examination;
an endoscopic image acquisition apparatus (32) that acquires a time-series endoscopic image of the endoscopic examination as an implementation record of the endoscopic examination;
an image identification apparatus (34) that detects a state of the examination target organ based on an endoscopic image corresponding to the predetermined examination step included in the time-series endoscopic image; and
an examination time period classification apparatus (36) that classifies the state of the examination target organ detected by the image identification apparatus (34) and calculates an actual time for each state in the predetermined examination step based on a classification result for each state.

2. The endoscopic examination support system according to claim 1, wherein
the examination time period evaluation apparatus (37) compares the evaluation index for each state with the actual time for each state and evaluates the actual time for each state.

3. The endoscopic examination support system according to claim 2,
further comprising a display controller (38) that displays, on a display apparatus (41), any one of the evaluation index for each state, the actual time for each state, and a comparison result between the evaluation index for each state and the actual time for each state from the examination time period evaluation apparatus (37).

4. The endoscopic examination support system according to claim 1, wherein
the examination time period classification apparatus (36) calculates an accumulated time length for each state of the examination target organ based on a classification result for each state, and
the examination time period evaluation apparatus (37) sets the evaluation index for each state in accordance with the accumulated time length for each state.

5. The endoscopic examination support system according to claim 1, wherein
the examination time period classification apparatus (36) calculates an accumulated time length for each state of the examination target organ based on a classification result for each state, and
the examination time period evaluation apparatus (37) sets the evaluation index for each state in accordance with the accumulated time length for each state and the type of the state of the examination target organ.

6. The endoscopic examination support system according to claim 1, wherein
the state of the examination target organ detected by the image identification apparatus (34) includes a state of a fold in the intestinal tract.

7. The endoscopic examination support system according to claim 1, wherein
the state of the examination target organ detected by the image identification apparatus (34) includes the presence or absence of diverticula in the intestinal tract.

8. An endoscopic examination support method comprising the steps of:
causing an examination time period evaluation apparatus (37) to acquire an evaluation index for each state in which a time evaluation index related to a predetermined examination step in an endoscopic examination is defined for each state of an examination target organ in the endoscopic examination;
causing an endoscopic image acquisition apparatus (32) to acquire a time-series endoscopic image of the endoscopic examination as an implementation record of the endoscopic examination;
causing an image identification apparatus (34) to detect a state of the examination target organ based on an endoscopic image corresponding to the predetermined examination step included in the time-series endoscopic image; and
causing an examination time period classification apparatus (36) to classify the state of the examination target organ detected by the image identification apparatus (34) and calculate an actual time for each state in the predetermined examination step based on a classification result for each state.

9. The endoscopic examination support method according to claim 8, wherein
the examination time period evaluation apparatus (37) compares the evaluation index for each state with the actual time for each state and evaluates the actual time for each state.

10. The endoscopic examination support method according to claim 9, wherein
a display controller (38) displays, on a display apparatus (41), any one of the evaluation index for each state, the actual time for each state, and a comparison result between the evaluation index for each state and the actual time for each state from the examination time period evaluation apparatus (37).

11. The endoscopic examination support method according to claim 8, wherein
the examination time period classification apparatus (36) calculates an accumulated time length for each state of the examination target organ based on a classification result for each state, and
the examination time period evaluation apparatus (37) sets the evaluation index for each state in accordance with the accumulated time length for each state.

12. The endoscopic examination support method according to claim 8, wherein
the examination time period classification apparatus (36) calculates an accumulated time length for each state of the examination target organ based on a classification result for each state, and
the examination time period evaluation apparatus (37) sets the evaluation index for each state in accordance with the accumulated time length for each state and the type of the state of the examination target organ.

13. The endoscopic examination support method according to claim 8, wherein
the state of the examination target organ detected by the image identification apparatus (34) includes a state of a fold in the intestinal tract.

14. The endoscopic examination support method according to claim 8, wherein
the state of the examination target organ detected by the image identification apparatus (34) includes the presence or absence of diverticula in the intestinal tract.

15. A storage medium storing a program for causing a computer to execute:
a process of causing an examination time period evaluation apparatus (37) to acquire an evaluation index for each state in which a time evaluation index related to a predetermined examination step in an endoscopic examination is defined for each state of an examination target organ in the endoscopic examination;
a process of causing an endoscopic image acquisition apparatus (32) to acquire a time-series endoscopic image of the endoscopic examination as an implementation record of the endoscopic examination;
a process of causing an image identification apparatus (34) to detect a state of the examination target organ based on an endoscopic image corresponding to the predetermined examination step included in the time-series endoscopic image; and
a process of causing an examination time period classification apparatus (36) to classify the state of the examination target organ detected by the image identification apparatus (34) and calculate an actual time for each state in the predetermined examination step based on a classification result for each state.

16. The storage medium according to claim 15, wherein
the storage medium stores a program for causing the examination time period evaluation apparatus (37) to execute a process of comparing the evaluation index for each state with the actual time for each state and evaluating the actual time for each state.

17. The storage medium according to claim 16, wherein
the storage medium stores a program for causing a computer to execute a process of causing a display controller (38) to display, on a display apparatus (41), any one of the evaluation index for each state, the actual time for each state, and a comparison result between the evaluation index for each state and the actual time for each state from the examination time period evaluation apparatus (37).

18. The storage medium according to claim 15, wherein
the storage medium stores a program for causing a computer to execute a process of causing the examination time period classification apparatus (36) to calculate an accumulated time length for each state of the examination target organ based on a classification result for each state, and
causing the examination time period evaluation apparatus (37) to set the evaluation index for each state in accordance with the accumulated time length for each state.

19. The storage medium according to claim 15, wherein
the storage medium stores a program for causing a computer to execute a process of causing the examination time period classification apparatus (36) to calculate an accumulated time length for each state of the examination target organ based on a classification result for each state, and
causing the examination time period evaluation apparatus (37) to set the evaluation index for each state in accordance with the accumulated time length for each state and the type of the state of the examination target organ.
